(19) United States
(12) Patent Application Publication
(10) Pub. No.: US 2025/0375540 A1
(43) Pub. Date: Dec. 11, 2025

(54) FIBROBLAST ACTIVATION PROTEIN TARGETING PRECURSOR AND RADIOTRACER

(71) Applicant: ITM ONCOLOGICS GMBH, Garching/Munich (DE)

(72) Inventors: Frank RÖSCH, Berlin (DE); Marian MECKEL, Freising (DE); Euy Sung MOON, Mainz (DE)

(21) Appl. No.: 18/877,137

(22) PCT Filed: Jun. 22, 2023
(86) PCT No.: PCT/EP2023/066972

§ 371 Date: Dec. 19, 2024

(30) Foreign Application Priority Data
Jun. 28, 2022 (DE) 10 2022 116 009.2

(51) Int. Cl. A61K 51/04; C07B 59/00

(52) U.S. Cl. CPC A61K 51/0455; A61K 51/0497; C07B 59/002; A61K 2123/00; C07B 2200/05

(57) ABSTRACT

A precursor compound and radiotracer for targeting fibroblast activation protein comprise a chelator Ch and ligand TV with Ch=(Formula) with R═|—CH3 or |—CH2COOH; and TV=(Formula).

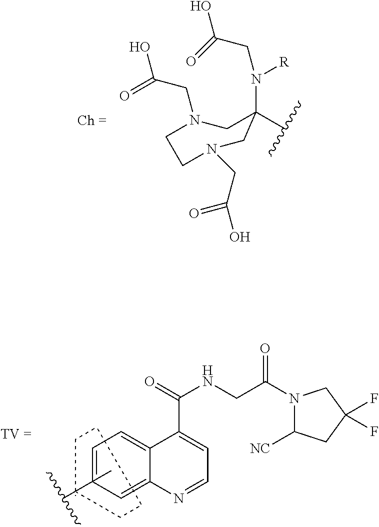

### SUMMARY

The present invention pertains to a precursor compound and therefrom derived radiotracer for imaging expression of fibroblast activation protein (FAP). The inventive precursor compound has the structure Ch-L1-B-L2-TV, wherein

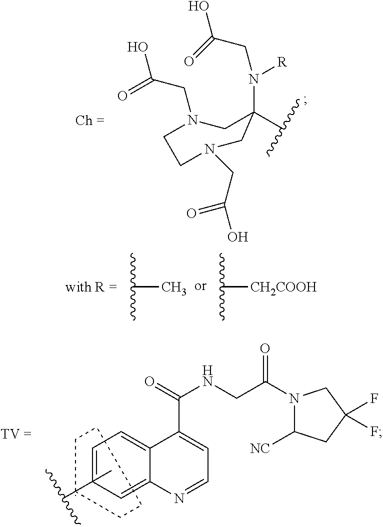

L1 is a bifunctional linker moiety;
L2 is a bifunctional linker moiety;
B is absent or a bifunctional linker moiety;
L1 is configured to covalently bind to Ch and L2 or to Ch and B, if B is present;
B, if present, is configured to covalently bind to L1 and to L2; and
L2 is configured to covalently bind to L1 and TV or to TV and B, if B is present.

### BACKGROUND

### Nuclear Medicine Diagnosis of Tumours with ⁶⁸Ga

Positron Emission Tomography (PET) combined with Computed Tomography (CT) using Gallium-68 (Ga-68 or ⁶⁸Ga) is today a well-established nuclear medicine examination. The U.S. Food and Drug Administration as well as the European Medicinal Agency approved Ga-68 labelled 1,4,7,10-tetraazacyclododecane-1,4,7,10-tetraacetic acid (DOTA)-octreotate (DOTATATE) and DOTA(0)-d-Phe(1)-Tyr(3)-octreotide, or DOTATOC for localization of somatostatin receptor positive neuroendocrine tumours (NETs) in adult and paediatric patients (in US) and for adult patients who are thought to have so-called well-differentiated gastroenteropancreatic neuroendocrine tumours (GEP-NETs) (in EU). Recently prostate specific membrane antigen inhibitor PSMA-11 was approved for imaging and staging of metastasized prostate carcinomas. The diagnostic value of PET/CT is mainly driven by sensitivity, specificity and accuracy. Sensitivity measures the proportion of positives that are correctly identified (true-positives divided by the sum of true-positives and false-negatives). Specificity measures the proportion of negatives that are correctly identified (true-negatives divided by the sum of true-negatives and false-positives). Diagnostic accuracy relates to the ability of a test to discriminate between the target condition and health. This discriminative potential can be quantified by the measures of sensitivity and specificity, target to background ratios, or the area under the receiver operating characteristic curve (ROC curve).

Fibroblast activation protein (FAP), a membrane-bound serine protease, is currently gaining increasing attention in nuclear medicine for targeting different cancer types with FAP-specific radiolabeled agents for both diagnostics and therapy. FAP is overexpressed by cancer associated fibroblasts (CAFs) which are abundant in more than 90% of common human epithelial tumors. CAFs are a myofibroblastic phenotype and situated in the tumor stroma. Stromal cells comprise up to 90% of the total tumor mass. FAP may also be expressed by fibroblasts present in wound healing, chronic inflammation, liver cirrhosis, rheumatoid arthritis, pulmonary fibrosis, bone and soft tissue sarcomas. However, in healthy tissue is FAP expression is practically absent. Hence, FAP-targeting radiopharmaceuticals are well suited for molecular imaging via PET or SPECT as well as radioendotherapy of cancer tumors and metastases.

### Quinoline-glycine-4,4-difluoro-2-cyanopyrrolidine Inhibitor of FAP

A potent FAP inhibitor (FAPi), commonly designated as UAMC1110, is based on a quinoline-glycine-4,4-difluoro-2-cyanopyrrolidine scaffold. UAMC1110 has low nanomolar FAP affinity and high selectivity with respect to enzymes of the dipeptidyl peptidase (DPP) and prolyl oligopeptidase (PREP) family. The high FAP-selectivity of UAMC1110 is particularly attractive for tumor-targeting, when considering the near-ubiquitous expression of DPP and PREP in the human body. As PREP displays the same endopeptidase activity as FAP and is abundant in healthy organs and tissues, high FAP specificity is essential for potent FAP ligands. UAMC1110 exhibits FAP affinity in the low nM range and PREP and DPP4 affinity in the μM range. Hence, UAMC1110 based FAPi-radiopharmaceuticals have recently been applied in nuclear medicine.

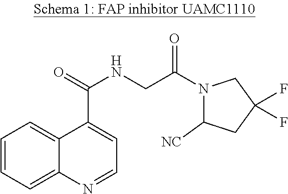 [SMILES: N#CC1CC(F)(F)CN1C(=O)CNC(=O)c1ccnc2ccccc12]

### Chelators in Radiometal-Containing Radiopharmaceuticals

The following properties are relevant for a precursor and therefrom derived radiotracer:
fast and effective complexation of a diagostic or therapeutic radioisotope to the labeling group;
high affinity and selectivity for tumor cells and metastases relative to healthy tissue;
in vivo stability, i.e. biochemical persistence in blood serum under physiological conditions;
high uptake in tumors and metastases for precise diagnosis and effective therapy;
rapid clearance from healthy tissues and blood to minimize systemic dose and toxicity.

According to current knowledge in the art:
the chelator and radioisotope greatly influence affinity and pharmacokinetics of radiotracers;
DOTA can severely affect the affinity of a targeting ligand;
chelator, radioisotope and targeting ligand interact unpredictably in synergistic or antagonistic manner.

Fani et al. report that the chelator moiety and the radiometal of a radiotracer can strongly affect the affinity of a targeting ligand. Reubi et al. show that conjugation of a targeting ligand with DOTA can substantially reduce its affinity to the targeted cellular receptor. The data presented by Reubi et al. indicates that a combination of chelator, radiometal and targeting ligand can yield a synergistic or antagonistic effect which are rarely predictable. In general conjugation of a targeting ligand with a linker and a large chelator moiety is non-beneficial and severly mutes affinity to the targeted cellular receptor.

The chelator DOTA, for example, is not well suited for complexing the relatively small (radio) metal Gallium and necessitates elevated reaction temperature which is detrimental for many antibodies and heat-sensitive biomolecules. After complexation ⁶⁸Ga-DOTA chelates require time for cooling prior to intravenous injection, thereby imposing limitations for clinical use due to the short ⁶⁸Ga half-life of 67.7 min.

### “Hybrid” Chelator Scaffolds DATA and AAZTA

DATA (1,4-bis(carboxymethyl)-6-[methyl-carboxymethyl-amino]-1,4-diazepane) and AAZTA (1,4-bis(carboxymethyl)-6-[bis(carboxymethyl)-amino]-1,4-diazepane) exhibit cyclic, acyclic and hybrid properties and has been found to be provide favorable labeling characteristics for ⁶⁸Ga. In particular, DATA enables fast and quantitative ⁶⁸Ga-labelling in a wide pH range at ambient temperature. Furthermore, [⁶⁸Ga] Ga-DATA chelates are immune against trans-chelation (DTPA and apo-transferrin) and trans-metalation (Fe^{III}) and stable in physiological milieu.

### DETAILED DESCRIPTION

The invention has the object to improve nuclear theranostics of endothelial cancer tumors and metastases that exhibit elevated FAP expression.

This object is achieved by a precursor compound having structure Ch-L1-B-L2-TV, wherein

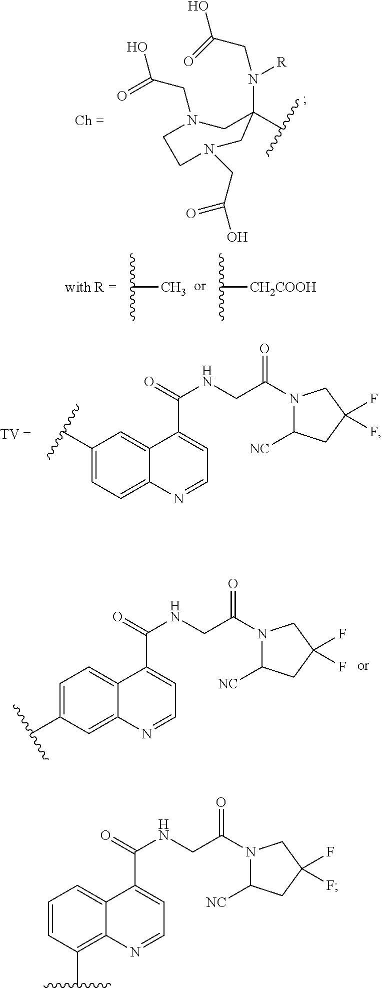

L1 is a bifunctional linker moiety;
L2 is a bifunctional linker moiety;
B is absent or a bifunctional linker moiety;
L1 is configured to covalently bind to Ch and L2 or to Ch and B, if B is present;
B, if present, is configured to covalently bind to L1 and to L2; and
L2 is configured to covalently bind to L1 and TV or to TV and B, if B is present.

Expedient embodiments of the inventive precursor compound are characterized by one of the following features or a combination of two or more of the following features insofar the combined features are not mutually exclusive or contradictory and according to which:

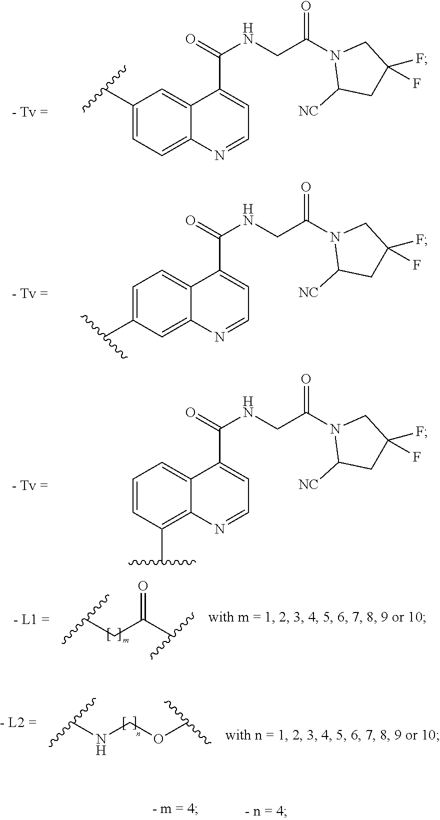

L1 has the structure

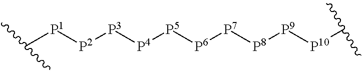

wherein
   each Pⁱ is present for 1≤i≤k and absent for i>k with k=1, 2, 3, 4, 5, 6, 7, 8, 9 or 10;
   each P^{s} with s=1, 2, 3, 4, 5, 6, 7, 8, 9 or 10, if present, independently from one another is selected from the group comprising —CH₂—, —CH₂CH₂O—, —N(H)—, —C(O)— and —C(CH₃)—;
L2 has the structure

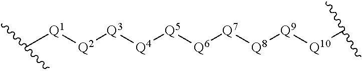

wherein
   each Q is present for 1≤j≤h and absent for j>h with h=1, 2, 3, 4, 5, 6, 7, 8, 9 or 10;
   each Q⁵ with s =1, 2, 3, 4, 5, 6, 7, 8, 9 or 10, if present, independently from one another is selected from the group comprising —CH₂—, —CH₂CH₂O—, —N(H)—, —C(O)— and —C(CH₃)—;
      absent

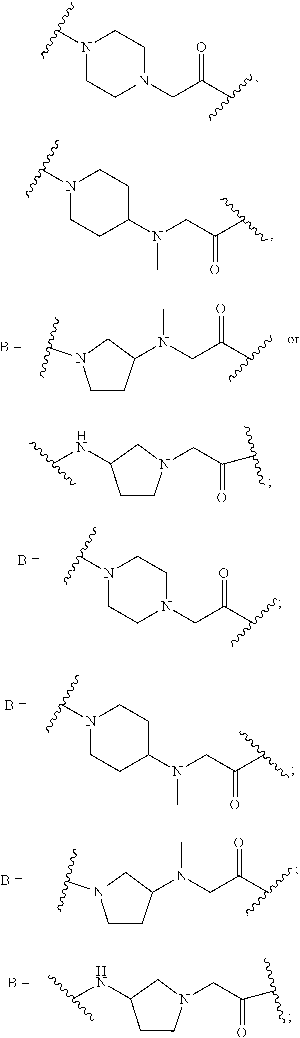

B is absent;
the precursor compound has the structure

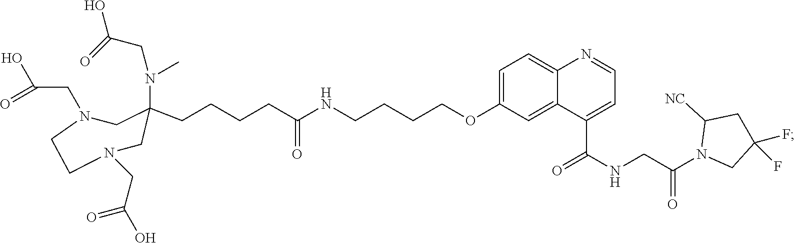 [SMILES: CN(CC(=O)O)C1(CCCCC(=O)NCCCCOc2ccc3nccc(C(=O)NCC(=O)N4CC(F)(F)CC4C#N)c3c2)CN(CC(=O)O)CCN(CC(=O)O)C1]

the precursor compound has the structure

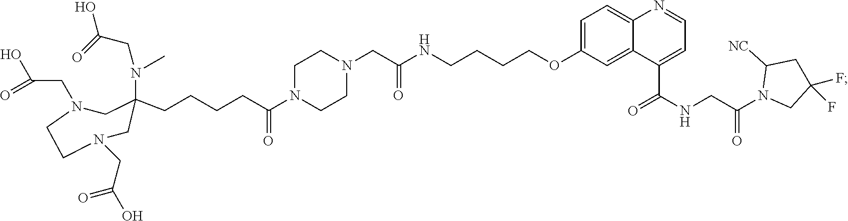 [SMILES: CN(CC(=O)O)C1(CCCCC(=O)N2CCN(CC(=O)NCCCCOc3ccc4nccc(C(=O)NCC(=O)N5CC(F)(F)CC5C#N)c4c3)CC2)CN(CC(=O)O)CCN(CC(=O)O)C1]

the precursor compound has the structure

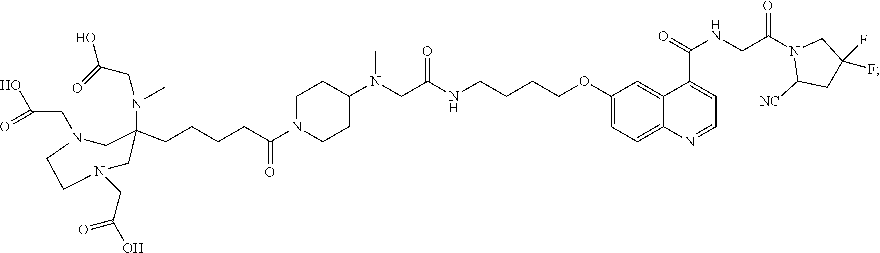 [SMILES: CN(CC(=O)NCCCCOc1ccc2nccc(C(=O)NCC(=O)N3CC(F)(F)CC3C#N)c2c1)C1CCN(C(=O)CCCCC2(N(C)CC(=O)O)CN(CC(=O)O)CCN(CC(=O)O)C2)CC1]

the precursor compound has the structure

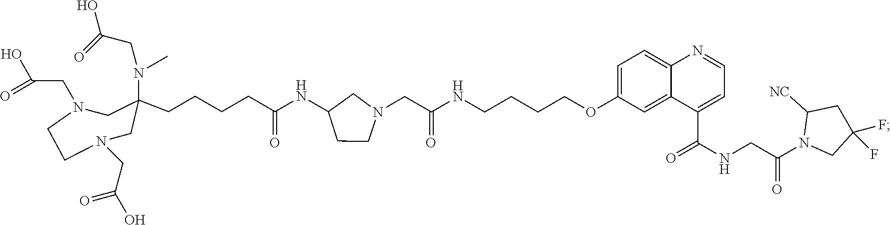 [SMILES: CN(CC(=O)O)C1(CCCCC(=O)NC2CCN(CC(=O)NCCCCOc3ccc4nccc(C(=O)NCC(=O)N5CC(F)(F)CC5C#N)c4c3)C2)CN(CC(=O)O)CCN(CC(=O)O)C1]

the precursor compound has the structure

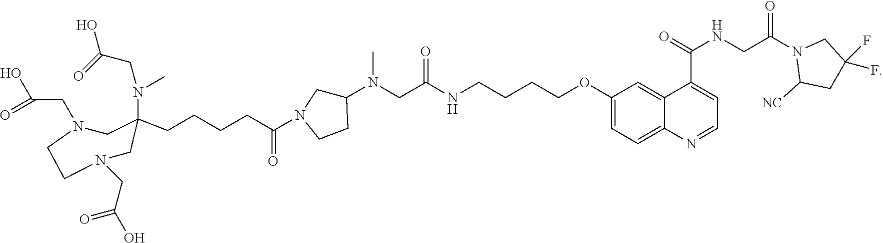 [SMILES: CN(CC(=O)NCCCCOc1ccc2nccc(C(=O)NCC(=O)N3CC(F)(F)CC3C#N)c2c1)C1CCN(C(=O)CCCCC2(N(C)CC(=O)O)CN(CC(=O)O)CCN(CC(=O)O)C2)C1]

The invention has the further object to provide a radiotracer that affords improved nuclear theranostics of endothelial cancer tumors and metastases with elevated FAP expression.

This object is achieved through a radiotracer comprised of the above described precursor compound and therewith complexed radioisotope ⁶⁸Ga.

Further expedient embodiments of the invention pertain to:
a radiopharmaceutical kit comprising one of the above described precursor compounds or a salt thereof;
a radiopharmaceutical kit comprising one of the above described precursor compounds or a salt thereof and a solvent selected from the group comprising water, 0.45% aqueous NaCl solution, 0.9% aqueous NaCl solution, Ringer solution (Ringer lactate), 5% aqueous dextrose solution and aqueous alcohol solution;
a radiopharmaceutical kit comprising
   a vial containing one of the above described precursor compounds or a salt thereof;
a radiopharmaceutical kit comprising
   a vial containing one of the above described precursor compounds or a salt thereof and a lyophilized buffering agent selected from the group comprising sodium acetate, gentisic acid, ascorbic acid, succinic acid, HEPES, sodium carbonate, sodium bicarbonate and salts thereof;
a radiopharmaceutical kit comprising
   a first vial containing one of the above described precursor compounds or a salt thereof, and
   a second vial containing
      a solvent selected from the group comprising water, 0.45% aqueous NaCl solution, 0.9% aqueous NaCl solution, Ringer solution (Ringer lactate), 5% aqueous dextrose solution and aqueous alcohol solution, or
      a buffer solution configured to modulate the pH between 3 to 7.4.

In this invention following synonymous compound designations are employed:

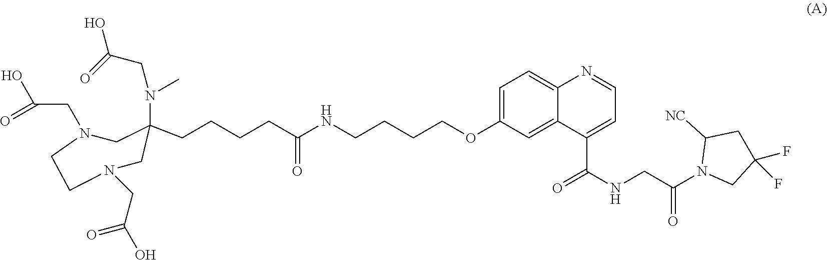 [SMILES: CN(CC(=O)O)C1(CCCCC(=O)NCCCCOc2ccc3nccc(C(=O)NCC(=O)N4CC(F)(F)CC4C#N)c3c2)CN(CC(=O)O)CCN(CC(=O)O)C1]

### DATA^{5m}.NH.FAPi

### (S)-2,2′-(6-((carboxymethyl)(methyl)amino)-6-(5-((4-((4-((2-(2-cyano-4,4-difluoropyrrolidin-1-yl)-2-oxoethyl)carbamoyl)quinolin-6-yl)oxy)butyl)amino)- 5-oxopentyl)-1,4-diazepane-1,4-diyl)diacetic Acid

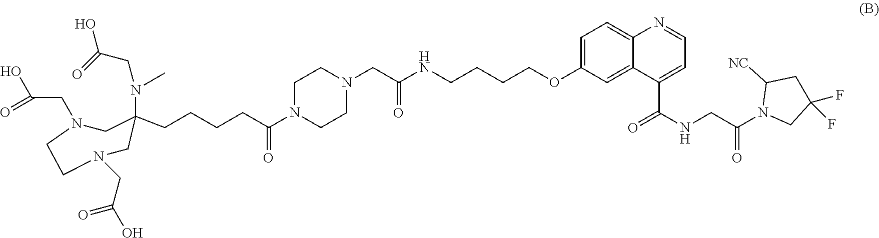 [SMILES: CN(CC(=O)O)C1(CCCCC(=O)N2CCN(CC(=O)NCCCCOc3ccc4nccc(C(=O)NCC(=O)N5CC(F)(F)CC5C#N)c4c3)CC2)CN(CC(=O)O)CCN(CC(=O)O)C1]

### DATA^{5m}.Pipz.FAPi

### (S)-2,2′-(6-((carboxymethyl)(methyl)amino)-6-(5-(4-(2-((4-((4-((2-(2-cyano-4,4-difluoropyrrolidin-1-yl)-2-oxoethyl)carbamoyl)quinolin-6- yl)oxy)butyl)amino)-2-oxoethyl)piperazin-1-yl)-5-oxopentyl)-1,4-diazepane-1,4-diyl)diacetic Acid

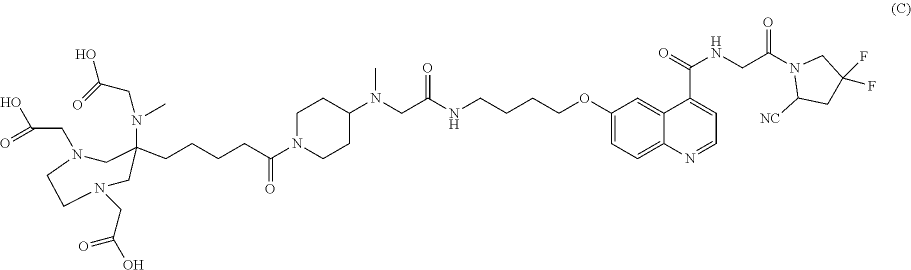 [SMILES: CN(CC(=O)NCCCCOc1ccc2nccc(C(=O)NCC(=O)N3CC(F)(F)CC3C#N)c2c1)C1CCN(C(=O)CCCCC2(N(C)CC(=O)O)CN(CC(=O)O)CCN(CC(=O)O)C2)CC1]

### DATA^{5m}.Pipd-N-Me.FAPi

### (S)-2,2′-(6-((carboxymethyl)(methyl)amino)-6-(5-(4-((2-((4-((4-((2-(2-cyano-4,4-difluoropyrrolidin-1-yl)-2-oxoethyl)carbamoyl)quinolin-6- yl)oxy)butyl)amino)-2-oxoethyl)(methyl)amino)piperidin-1-yl)-5-oxopentyl)-1,4-diazepane-1,4-diyl)diacetic Acid

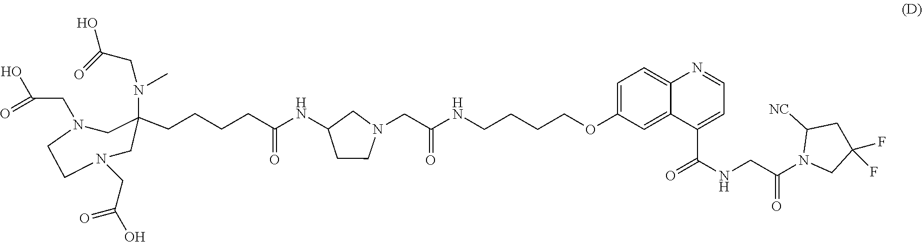 [SMILES: CN(CC(=O)O)C1(CCCCC(=O)NC2CCN(CC(=O)NCCCCOc3ccc4nccc(C(=O)NCC(=O)N5CC(F)(F)CC5C#N)c4c3)C2)CN(CC(=O)O)CCN(CC(=O)O)C1]

### DATA^{5m}.NH-Pyr.FAPi

### 2,2′-(6-((carboxymethyl)(methyl)amino)-6-(5-(((S)-1-(2-((4-((4-((2- ((S)-2-cyano-4,4-difluoropyrrolidin-1-yl)-2-oxoethyl)carbamoyl)quinolin-6-yl)oxy)butyl) amino)-2-oxoethyl)pyrrolidin-3-yl)amino)-5-oxopentyl)-1,4-diazepane-1,4-diyl)diacetic Acid

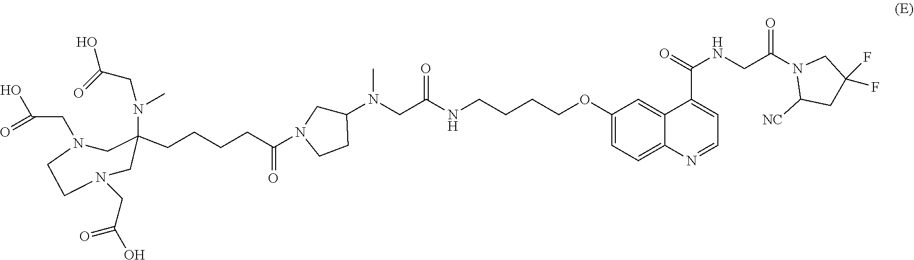 [SMILES: CN(CC(=O)NCCCCOc1ccc2nccc(C(=O)NCC(=O)N3CC(F)(F)CC3C#N)c2c1)C1CCN(C(=O)CCCCC2(N(C)CC(=O)O)CN(CC(=O)O)CCN(CC(=O)O)C2)C1]

### DATA^{5m}.Pyr-N-Me.FAPi

### 2,2′-(6-((carboxymethyl)(methyl)amino)-6-(5-((S)-3-((2-((4-((4-((2-((S)-2-cyano-4,4-difluoropyrrolidin-1-yl)-2-oxoethyl)carbamoyl)quinolin-6-yl)oxy)butyl)amino)-2-oxoethyl)(methyl)amino)pyrrolidin-1-yl)-5-oxopentyl)-1,4-diazepane-1,4-diyl)diacetic Acid

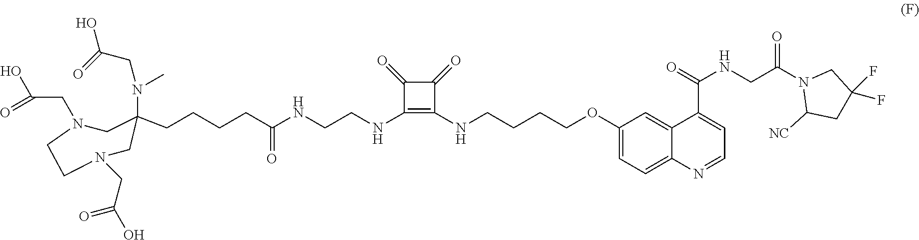 [SMILES: CN(CC(=O)O)C1(CCCCC(=O)NCCNc2c(NCCCCOc3ccc4nccc(C(=O)NCC(=O)N5CC(F)(F)CC5C#N)c4c3)c(=O)c2=O)CN(CC(=O)O)CCN(CC(=O)O)C1]

### DATA^{5m}.SA.FAPi

### 2,2′-(6-((carboxymethyl)(methyl)amino)-6-(5-((S)-3-((2-((4-((4-((2-((S)-2-cyano-4,4-difluoropyrrolidin-1-yl)-2-oxoethyl)carbamoyl)quinolin-6-yl)oxy)butyl)amino)-2-oxoethyl)(methyl)amino)pyrrolidin-1-yl)-5-oxopentyl)-1,4-diazepane-1,4-diyl)diacetic Acid

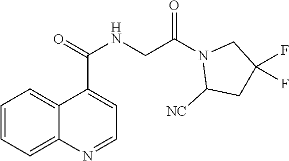 [SMILES: N#CC1CC(F)(F)CN1C(=O)CNC(=O)c1ccnc2ccccc12]

### UAMC1110

### (S)-N-(2-(2-cyano-4,4-difluoropyrrolidin-1-yl)-2-oxoethyl)quinoline-4-carboxamide

### EXAMPLES

### Example 1: DATA^{5m} Prochelator Synthesis

Schema 2 illustrates the synthesis of the DATA^{5m} prochelator (cf. J. Seemann, B. Waldron, D. Parker, F. Roesch; DATATOC: a novel conjugate for kit-type ⁶⁸Ga labelling of TOC at ambient temperature; EJNMMI Radiopharmacy and Chemistry (2016) 1:4, DOI 10.1186/s41181-016-0007-3).

### 5-(1,4-Dibenzyl-6-nitro-[1, 4]diazepan-6-yl)-pentanoic Acid Methyl Ester (1) 2-Nitrocyclohexanone (0.608 g, 4.3 mmol) is added to Amberlyst A21 (1.216 g, 2 mass equivalents) in EtOH and stirred for 2 h at 60° C. under argon. N,N′-Dibenzyl-ethylenediamine (1.020 g, 4.3 mmol) and paraformaldehyde (0.446 g, 14.9 mmol) were added and the reaction stirred at 60° C. overnight. The mixture is filtered through Celite®, and solvent removed under reduced pressure. The resulting residue is re-dissolved in CHCl₃ (40 ml) and washed successively with aqueous K₂CO₃ solution (2×30 mL, 0.1 M) and H₂O (30 mL), dried over MgSO₄, filtered and solvent removed under reduced pressure. Purification by silica gel column chromatography (DCM) afforded the title compound as a yellow oil (1.607 g, 85%). Rf=0.80 (DCM).

### 5-(1,4-Dibenzyl-6-nitro-[1,4]diazepan-6-yl)-pentanoic Acid Methyl Ester (2)

A catalytic amount of Pd(OH)₂/C and acetic acid (50 μL, 0.87 mmol) is added to the protected triamine 1 (0.10 g, 0.29 mmol) in MeOH (20 mL), and the mixture agitated under an atmosphere of hydrogen for 3 h (1 atm H₂). TLC (DCM) is used to confirm complete reduction of the nitro group and cleavage of the benzyl N-substituents. Pd(OH)₂/C is removed using a Celite® filter. The solvent is removed under reduced pressure to afford a yellow oil (0.065 g, 97%).

### 5-[1,4-Bis-tert-butoxycarbonylmethyl-6-(tert-butoxycarbonylmethyl-amino)-[1,4]diazepan-6-yl]-pentanoic Acid Methyl Ester (3)

tert-Butyl-bromoacetate (0.567 g, 2.91 mmol) is added to 2 (0.208 g, 0.91 mmol) and K₂CO₃ (0.377 g, 2.73 mmol) in MeCN (25 mL), and the mixture stirred for 24 h at 368 K under argon atmosphere. The reaction is monitored by TLC (hexane/ethyl acetate; 1:1) for formation of the tetraalkylated derivative. The solvent is removed under reduced pressure, and the resulting oil re-dissolved in CHC₃ (25 mL) and washed successively with aqueous K₂CO₃ solution (2×25 mL, 0.1 M) and H₂O (25 mL), dried over MgSO₄, filtered and the solvent removed under reduced pressure. Purification by silica gel column chromatography (hexane/ethyl acetate, 2:1→1:1) affords a yellow oil (0.229 g, 44%). Rf=0.35 (hexane/ethyl acetate; 2:1).

### 5-[1,4-Bis-tert-butoxycarbonylmethyl-6-(tert-butoxycarbonylmethyl-methyl-amino)-[1,4]diazepan-6-yl]-pentanoic Acid Methyl Ester (4)

Iodomethane (0.023 g, 0.16 mmol) is added to 3 (0.104 g, 0.18 mmol) and K₂CO₃ (0.025 g, 0.18 mmol) in DCM/MeCN (3:1) cooled in an ice-bath. The reaction mixture is allowed to warm to room temperature and left overnight. The solvent is removed under reduced pressure and the resulting oil re-dissolved in CHCl₃ (20 mL), filtered and washed successively with aqueous K₂CO₃ solution (2×20 mL, 0.1 M) and H₂O (20 mL), dried over MgSO₄, filtered and solvent removed under reduced pressure. Purification by silica gel column chromatography (hexane/ethyl acetate, 3:1→2:1) afforded a yellow oil (0.043 g, 46%). Rf=0.38 (hexane/ethyl acetate; 2:1).

### 5-[1,4-Bis-tert-butoxycarbonylmethyl-6-(tert-butoxycarbonylmethyl-methyl-amino)-[1,4]diazepan-6-yl]-pentanoic Acid (5)

LiOH (0.009 g, 0.039 mmol) dissolved in H₂O (0.5 mL) is added to 4 (0.010 g, 0.023 mmol) in THF (0.5 mL), and the mixture stirred at 298 K. The reaction is monitored using LC-ESI MS for ester cleavage. Once complete, the solvent is removed by lyophilisation. H₂O (5 mL) is added and removed by lyophilisation and the procedure repeated two times. The resulting solid is washed with ice-cold DCM (0.5 mL), and dried in vacuo to yield a waxy yellow solid (0.009 g, 70%).

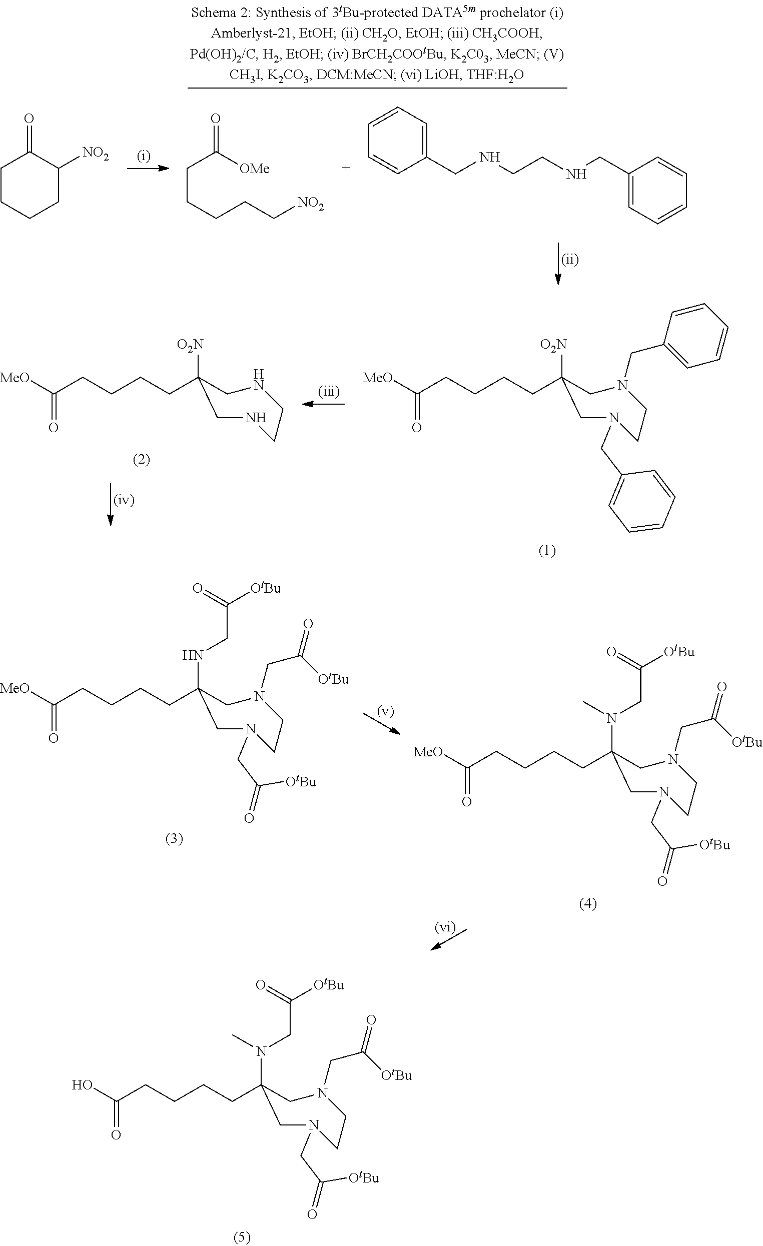

### Example 2: Synthesis of FAP Targeting Ligands

Schemata 3-5 illustrate the synthesis of FAP targeting ligands (cf. K. Jansen, L. Heirbaut, R. Verkerk, J.D. Cheng, J. Joossens, P. Cos, L. Maes, A.-M. Lambeir, I. De Meester, K. Augustyns, P. Van der Veken; Extended Structure-Activity Relationship and Pharmacokinetic Investigation of (4-Quinolinoyl) glycyl-2-cyanopyrrolidine Inhibitors of Fibroblast Activation Protein (FAP); J. Med. Chem. 2014 Apr. 10; 57 (7): 3053-74, DOI 10.1021/jm500031w).

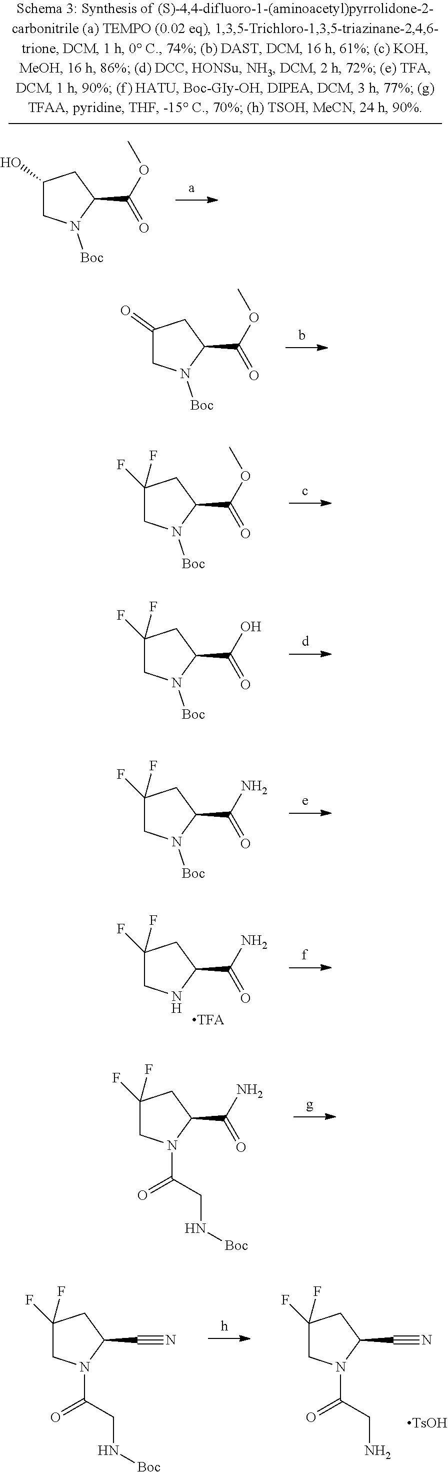

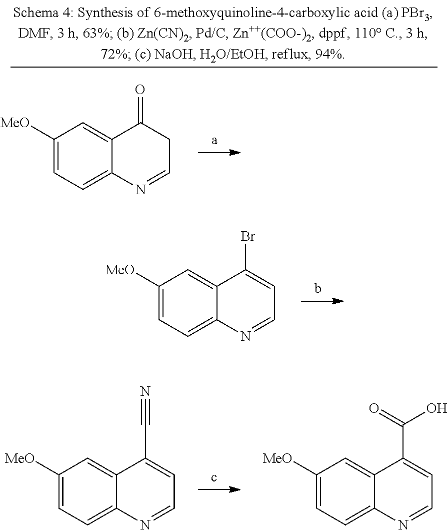 [SMILES: COc1ccc2c(c1)C(=O)CC=N2 ; COc1ccc2nccc(Br)c2c1 ; COc1ccc2nccc(C#N)c2c1 ; COc1ccc2nccc(C(=O)O)c2c1]

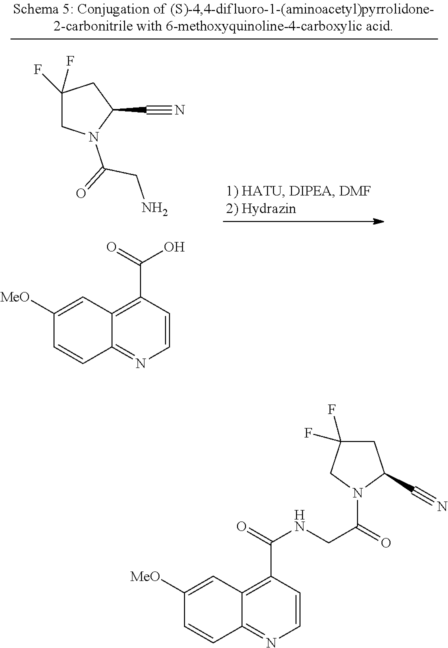

### Example 3: Affinity to FAP and PREP

The inventive precursor compounds and radiotracers exhibit high target affinity to FAP and excellent selectivity, i.e. low binding to PREP. Hence, they afford high image contrast for the diagnosis of endothelial tumors and metastases with Gallium-68 PET/CT.

Beneath Table 1 presents measured affinities to FAP and PREP for the inventive precursor compounds 1-4 and reference compound 5 as well as the original FAP inhibitor UAMC1110 as the current gold standard.

TABLE 1
Affinities to FAP and PREP

| | | PREP IC₅₀ | FAP IC₅₀ | | |
|---|---|---|---|---|---|
| # | Compound | [μM] | [nM] | S* | S_{F}** |
| (A) | DATA^{5m}•NH•FAPi | 6.4 ± 0.4 | 0.27 ± 0.02 | 23.7 × 10³ | 5.6 |
| (B) | DATA^{5m}•Pipz•FAPi | 1.5 ± 0.3 | 0.27 ± 0.01 | 5.6 × 10³ | 1.3 |
| (D) | DATA^{5m}•NH—Pyr•FAPi | 3.3 ± 0.2 | 0.28 ± 0.02 | 11.8 × 10³ | 2.8 |
| (E) | DATA^{5m}•Pyr—N—Me•FAPi | 2.6 ± 0.3 | 0.31 ± 0.02 | 8.4 × 10³ | 2.0 |
| (F) | DATA^{5m}•SA•FAPi | 1.7 ± 0.1 | 0.8 ± 0.02 | 2.1 × 10³ | 0.5 |
| (G) | UAMC1110 | 1.8 ± 0.01 | 0.43 ± 0.02 | 4.2 × 10³ | 1.0 |

*S = PREP IC₅₀/FAP IC₅₀
**S_{F} = S relative to UAMC1110

Inventive precursor compounds (A), (B), (D) and (E) show higher affinity to FAP than reference compound (F) and the UAMC1110 gold standard (G). Compound (A), (D) and (E) exhibit reduced binding to off-target PREP compared to reference (F) and (G). Compound (B) has PREP affinitiy comparable to (F) and (G). All of compounds (A), (B), (D), (E) have a distinctly higher selectivity for FAP than PREP compared to reference (F) and gold standard (G). The selectivity of compound (B) is similar to that of (G). The selectivity of compound (E) is twice that of (G). The selectivity of compound (D) is three times higher than that of (G). The selectivity of compound (A) is six times that of (G). The DATA^{5m}-conjugated reference compound (F) has lower affinity to FAP and lesser selectivity than (G) as expected due to chemical modification.

The inventive radiotracers can be conveniently provided as ready to use kit. Such kit contains one the above described freeze-dried precursor compound or a salt thereof, a buffer for pH adjustment, antioxidants as radical scavengers to prevent radiolysis and lyophilization bulking agents. The respective radiotracer is prepared by adding a hydrochloric acid solution of [⁶⁸Ga]GaCl₃ pursuant to European Pharmacopoeia monograph “Gallium (68Ga) chloride solution”.

### Example 4: PET MIP with [⁶⁸Ga]Ga-DATA^{5m}.Pip.FAPi

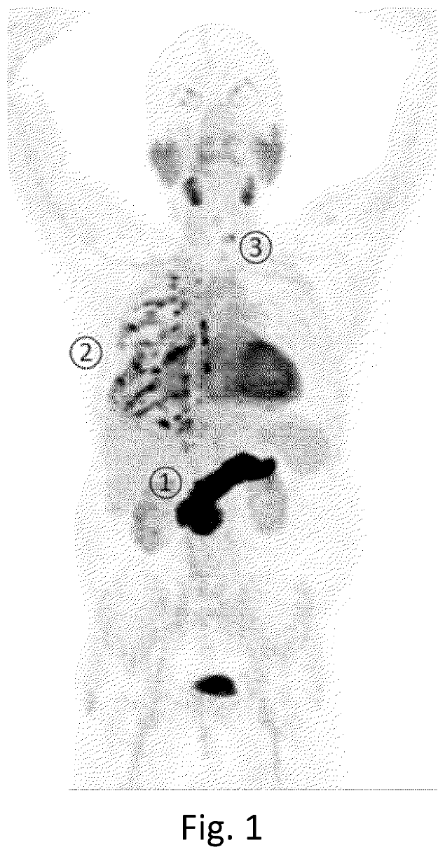

FIG. 1 shows an image acquired from a 52 year old male patient suffering from radioiodine refractory differentiated thyroid cancer via PET MIP using the inventive radiotracer [⁶⁸Ga]Ga-DATA^{5m}.Pip.FAPi. Multiple pancreas (1), lung (2) and lymph node (3) metastases are detected.

### Example 5: PET MIP with [⁶⁸Ga]Ga-DATA^{5m}.NH.FAPi

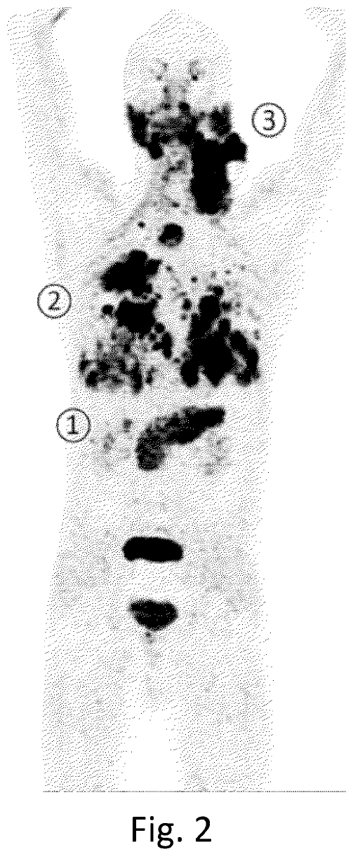

FIG. 2 shows an image acquired from a 40 year old male patient afflicted with radioiodine refractory differentiated thyroid cancer via PET MIP using the inventive radiotracer [⁶⁸Ga] Ga-DATA^{5m}.NH.FAPi. Multiple liver (1), lung (2) and lymph node (3) lesions are detected.

### Example 6: Coronal PET/CT Imaging

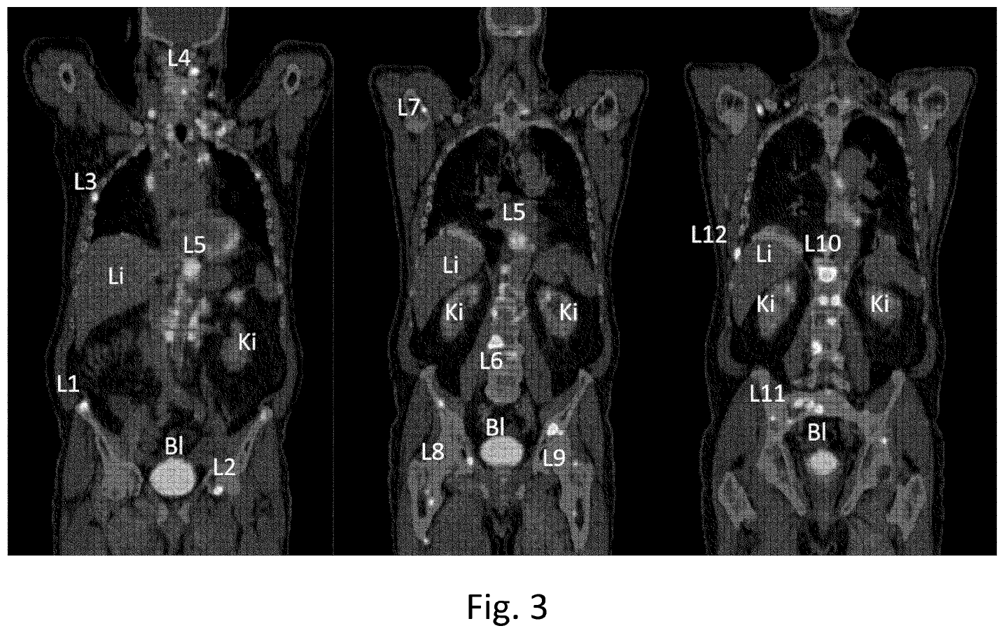

FIG. 3 depicts a coronal PET/CT patient image acquired from a male patient suffering from metastasized gastroesophageal cancer using the inventive radiotracer [⁶⁸Ga]Ga-DATA^{5m}.NH-Pyr.FAPi. Multiple skeletal metastases are detected

The annotations in FIG. 3 designate
Li: Liver
Ki: Kidney
BI: Bladder
L1: tumor bone lesion, pelvis
L2: tumor bone lesion, pelvis
L3: tumor bone lesion, rib
L4: tumor lesion
L5: tumor lesion
L6: tumor bone lesion, vertebra
L7: tumor bone lesion, shoulder
L8: multiple tumor bone lesion, pelvis
L9: tumor lesion, pelvis
L10: multiple tumor bone lesions, vertebra
L11: multiple tumor bone lesion, pelvis
L12: tumor bone lesion, rib

1. Precursor compound for radiotracer having structure Ch-L1-B-L2-TV, wherein 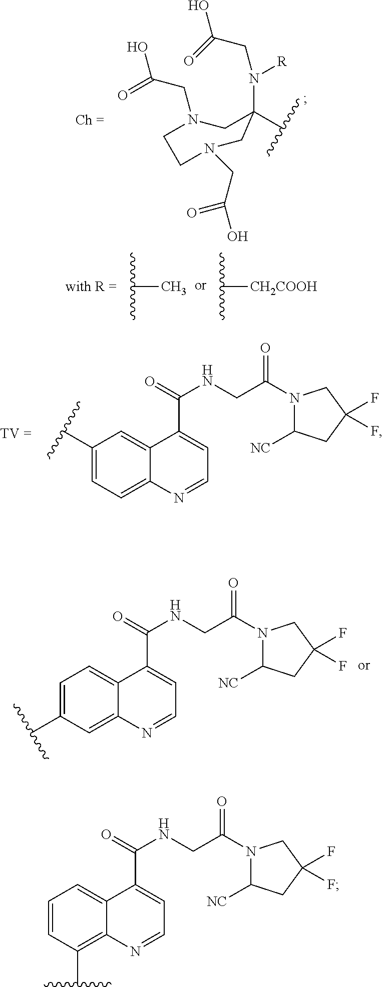
L1 is a bifunctional linker moiety;
L2 is a bifunctional linker moiety;
B is absent or a bifunctional linker moiety;
L1 is configured to covalently bind to Ch and L2 or to Ch and B, if B is present;
B, if present, is configured to covalently bind to L1 and to L2; and
L2 is configured to covalently bind to L1 and TV or to TV and B, if B is present.

2. Precursor compound according to claim 1, characterized in that

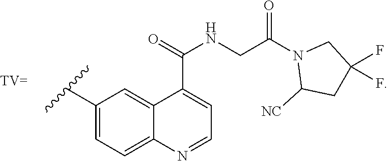 [SMILES: *c1ccc2nccc(C(=O)NCC(=O)N3CC(F)(F)CC3C#N)c2c1]

3. Precursor compound according to claim 1, characterized in that absent,

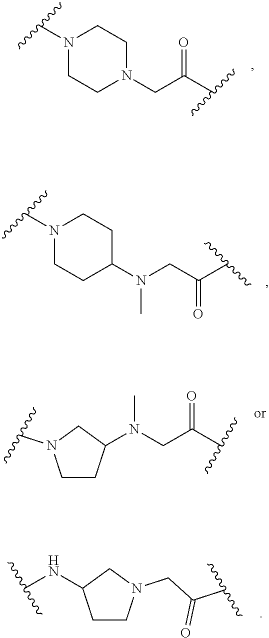 [SMILES: *C(=O)CN1CCN(*)CC1 ; *C(=O)CN(C)C1CCN(*)CC1 ; *C(=O)CN(C)C1CCN(*)C1 ; *NC1CCN(CC(*)=O)C1]

4. Precursor compound according to claim 1, characterized in that 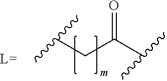
with m=1, 2, 3, 4, 5, 6, 7, 8, 9 or 10.

5. Precursor compound according to claim 4, characterized in that m=4.

6. Precursor compound according to claim 1, characterized in that 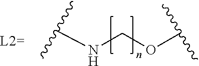
with n=1, 2, 3, 4, 5, 6, 7, 8, 9 or 10.

7. Precursor compound according to claim 6, characterized in that n=4.

8. Precursor compound according to claim 1, characterized in that

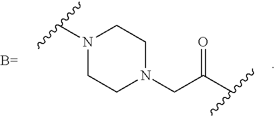 [SMILES: *C(=O)CN1CCN(*)CC1]

9. Precursor compound according to claim 1, characterized in that

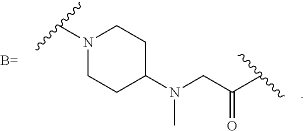 [SMILES: *C(=O)CN(C)C1CCN(*)CC1]

10. Precursor compound according to claim 1, characterized in that

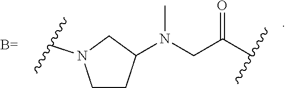 [SMILES: *C(=O)CN(C)C1CCN(*)C1]

11. Precursor compound according to claim 1, characterized in that

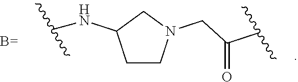 [SMILES: *NC1CCN(CC(*)=O)C1]

12. Precursor compound according to claim 1, characterized in that it has the structure

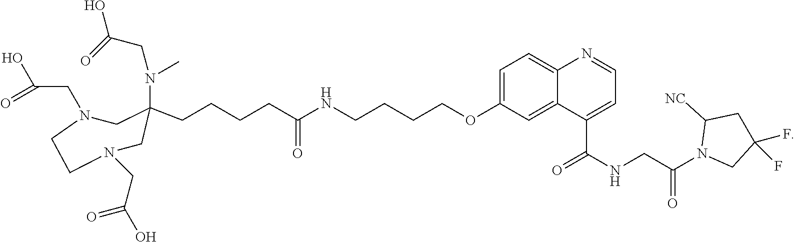 [SMILES: CN(CC(=O)O)C1(CCCCC(=O)NCCCCOc2ccc3nccc(C(=O)NCC(=O)N4CC(F)(F)CC4C#N)c3c2)CN(CC(=O)O)CCN(CC(=O)O)C1]

13. Precursor compound according to claim 1, characterized in that it has the structure

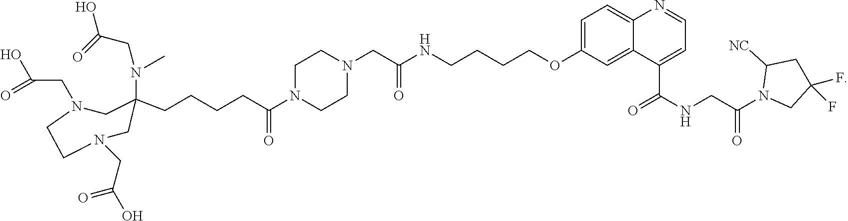 [SMILES: CN(CC(=O)O)C1(CCCCC(=O)N2CCN(CC(=O)NCCCCOc3ccc4nccc(C(=O)NCC(=O)N5CC(F)(F)CC5C#N)c4c3)CC2)CN(CC(=O)O)CCN(CC(=O)O)C1]

14. Precursor compound according to claim 1, characterized in that it has the structure

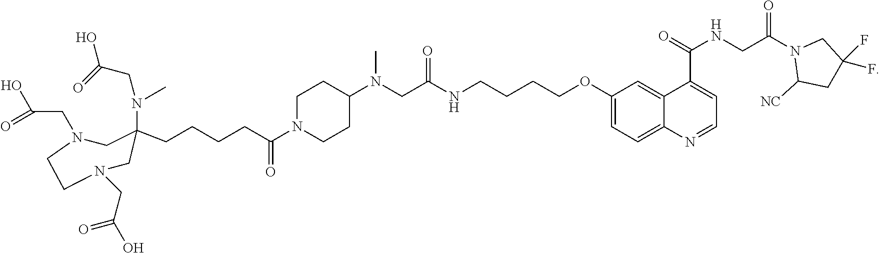 [SMILES: CN(CC(=O)NCCCCOc1ccc2nccc(C(=O)NCC(=O)N3CC(F)(F)CC3C#N)c2c1)C1CCN(C(=O)CCCCC2(N(C)CC(=O)O)CN(CC(=O)O)CCN(CC(=O)O)C2)CC1]

15. Precursor compound according to claim 1, characterized in that it has the structure

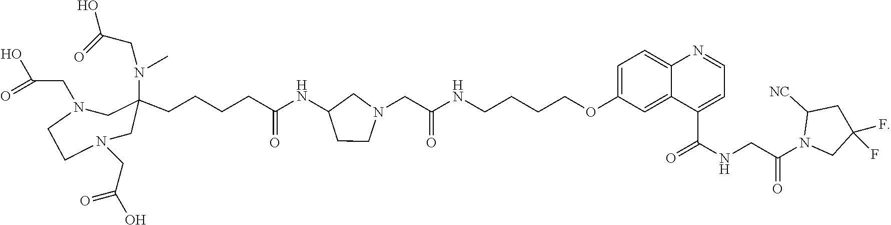 [SMILES: CN(CC(=O)O)C1(CCCCC(=O)NC2CCN(CC(=O)NCCCCOc3ccc4nccc(C(=O)NCC(=O)N5CC(F)(F)CC5C#N)c4c3)C2)CN(CC(=O)O)CCN(CC(=O)O)C1]

16. Precursor compound according to claim 1, characterized in that it has the structure

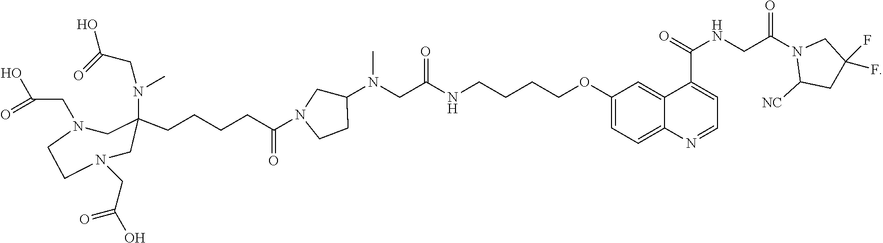 [SMILES: CN(CC(=O)NCCCCOc1ccc2nccc(C(=O)NCC(=O)N3CC(F)(F)CC3C#N)c2c1)C1CCN(C(=O)CCCCC2(N(C)CC(=O)O)CN(CC(=O)O)CCN(CC(=O)O)C2)C1]

17. Radiotracer comprised of the precursor compound according to claim 1 and therewith complexed radioisotope ⁶⁸Ga.
